(19) United States
(12) Patent Application Publication
(10) Pub. No.: US 2017/0135745 A1
(43) Pub. Date: May 18, 2017

(54) Liquid Ejection Device and Medical Apparatus

(71) Applicant: Seiko Epson Corporation, Tokyo (JP)

(72) Inventors: Hideki Kojima, Matsumoto-shi (JP); Hirokazu Sekino, Chino-shi (JP)

(21) Appl. No.: 15/129,741

(22) PCT Filed: Apr. 2, 2015
(86) PCT No.: PCT/JP2015/001895

§ 371 Date: Sep. 27, 2016

(30) Foreign Application Priority Data
Apr. 3, 2014 (JP) 2014-076592

(51) Int. Cl. A61B 18/14; A61B 18/12

(52) U.S. Cl. CPC A61B 18/14; A61B 18/1206; A61B 2017/32032

(57) ABSTRACT

A liquid ejection device includes an ejection tube, a gripping tube, a liquid chamber, a connection channel, and a pulsed-flow generating section. The ejection tube is a tube for ejecting liquid. The gripping section is an instrument attached to the ejection tube and gripped by a user. The liquid chamber is provided on the outside of the gripping section. The connection channel connects the liquid chamber and the ejection tube. The pulsed-flow generating section is provided on the outside of the gripping section and generates a pulsed flow in the liquid in the liquid chamber.

This application claims the benefit of Japanese Patent Application No. 2014-076592, filed on Apr. 3, 2014. The content of the aforementioned patent application is incorporated herein by reference in its entirety.

### TECHNICAL FIELD

The present invention relates to ejection of liquid.

### BACKGROUND ART

There is known a liquid ejection device that ejects liquid in a pulse-like manner to perform incision, excision, or the like of a target object (e.g., PTL 1). For example, in the medical field, the liquid ejection device is used as a medical apparatus that incises or excises a living tissue. The liquid ejection device includes a liquid chamber, the volume of which is changed by driving of a piezoelectric element. The liquid ejection device generates pulsation in a flow of the liquid to thereby eject the liquid from an ejection tube in a pulse-like manner at high speed. The ejection tube is attached to a gripping section. A user grips the gripping section and operates the ejection tube.

### CITATION LIST

### Patent Literature

[PTL 1]

JP-A-2008-82202

### SUMMARY OF INVENTION

### Technical Problem

A problem to be solved by the invention is to reduce the gripping section in weight. If the gripping section is reduced in weight, the operation of the gripping section is facilitated. In the related art, a device that generates a pulsed flow is incorporated in the gripping section. The related art has a problem in that the gripping section is heavy. Alternatively, the related art has a problem in that, when a function of an electric knife is added to the gripping section or a mechanism for sucking ejected liquid or the like is added to the gripping section, the gripping section is increased in weight because of structural interference with the device that generates a pulsed flow and electric protection measures. In particular, when the liquid ejection device is used as the medical apparatus, precise work is requested for a long time. Therefore, it has been desired to reduce the gripping section in weight. Besides, there have been demands for a reduction in the size, a reduction in costs, resource saving, facilitation of manufacturing, improvement of convenience of use, and the like of the device.

### Solution to Problem

An advantage of some aspects of the invention is to solve at least a part of the problem described above, and the invention can be implemented as the following aspects.

(1) An aspect of the invention provides a liquid ejection device. The liquid ejection device includes: an ejection tube for ejecting liquid; a gripping section attached to the ejection tube and gripped by a user; a liquid chamber provided on the outside of the gripping section; a connection channel configured to connect the liquid chamber and the ejection tube; and a pulsed-flow generating section provided on the outside of the gripping section and configured to generate a pulsed flow in the liquid in the liquid chamber. According to this aspect, since the liquid chamber and the pulsed-flow generating section are provided on the outside of the gripping section, the gripping section is reduced in weight.

(2) In the aspect described above, the liquid ejection device may further include a power supply device configured to supply high-frequency power to an electrode provided in the gripping section. According to this aspect, it is possible to impart a function of an electric knife to the gripping section. Moreover, since the liquid chamber and the pulsed-flow generating section is provided on the outside of the gripping section, it is unnecessary to provide, in the gripping section, a member used for electric protection measures for the liquid chamber and the pulsed-flow generating section. Therefore, even if the function of the electric knife is imparted to the gripping section, it is easy to avoid an increase in the weight of the gripping section.

(3) In the aspect described above, the gripping section may include a connection terminal to which a cable is detachably attachable. The connection terminal and an electrode provided in the gripping section may conduct to each other. According to this aspect, by supplying high-frequency power to the electrode via the connection terminal, it is possible to impart a function of an electric knife to the gripping section. In addition, as in the aspect explained above, it is easy to avoid an increase in the weight of the gripping section. Moreover, since the cable is detachably attachable, when the function of the electronic knife is not used, it is possible to attain a reduction in weight by detaching the cable. Note that a power supply device that supplies the high-frequency power may or may not be included in the liquid ejection device.

(4) In the aspect described above, the electrode may be the distal end of the ejection tube. According to this aspect, since it is unnecessary to separately provide the electrode, an increase in the weight of the gripping section is suppressed.

(5) In the aspect described above, the gripping section may include a suction tube for sucking the liquid. The electrode may be the distal end of the suction tube. According to this aspect, since a function for sucking the liquid is imparted to the gripping section and it is unnecessary to separately provide the electrode, an increase in the weight of the gripping section is suppressed.

(6) In the aspect described above, the gripping section may further include a member that conducts to the connection terminal and forms the electrode. According to this aspect, it is easy to avoid carbide (burnt deposit), which occurs when the function of the electric knife is used, from adhering to the distal ends of the ejection tube and the suction tube.

(7) In the aspect described above, the gripping section and the ejection tube may be detachably attachable. According to this aspect, it is possible to separately replace and clean the gripping section and the ejection tube.

(8) In the aspect described above, the gripping section may include a suction tube for sucking the liquid. According to this aspect, it is possible to impart, to the gripping section, a function for sucking the liquid. Moreover, since the liquid chamber and the pulsed-flow generating section are provided on the outside of the gripping section, structural interference between the liquid chamber and the pulsed-flow generating section and the suction tube and a suction channel does not occur in the gripping section. Therefore, even if the gripping section includes the sucking function, it is easy to avoid the gripping section from being complicated and increased in weight. In addition, since the inside of the gripping section can be simply configured, it is easy to avoid the suction tube and the suction channel from being formed in a largely curved shape. Consequently, clogging of the suction tube and the suction channel is suppressed.

(9) In the aspect described above, at least a part of the connection channel may be a channel formed as an inner tube of a double tube, an outer tube of the double tube may be at least apart of a suction channel detachably attached to an end portion of the suction tube, and any one of the ejection tube and the connection channel may project from an end portion of the outer tube of the double tube. According to this aspect, it is possible to separate the ejection tube and the suction tube by detaching the gripping section from the suction channel. Consequently, it is possible to easily execute a change in the inner diameter of the suction tube.

(10) In the aspect described above, the gripping section may include a vibration absorbing member that absorbs vibration from the connection channel. According to this aspect, vibration of the gripping section is suppressed.

(11) Another aspect of the invention provides a liquid ejection device including: an ejection tube for ejecting liquid, a gripping section gripped by a user being attachable to the ejection tube; a connection channel configured to connect a liquid chamber and the ejection tube; and a pulsed-flow generating section configured to generate a pulsed flow in the liquid in the liquid chamber. According to this aspect, it is easy to design the gripping section such that, in a state in which the gripping section is attached to the ejection tube, the liquid chamber and the pulsed-flow generating section are located on the outside of the gripping section, that is, the gripping section does not include the liquid chamber and the pulsed-flow generating section. Consequently, it is easy to design the attached gripping section light in weight. In addition, it is possible to separately replace and clean the gripping section and the ejection tube.

(12) In the aspect described above, the ejection tube may include a combining member for the attachment, and the distal end of the ejection tube may conduct to at least a part of a site where the combining member is in contact with the gripping section. According to this aspect, by supplying high-frequency power via the gripping section, it is possible to cause the distal end of the ejection tube as an electrode of an electric knife.

In the aspect described above, the liquid ejection device may further include a suction tube coupled to the ejection tube to suck liquid, the attachment may be implemented via the suction tube, the suction tube may include a combining member for the attachment, and the distal end of the suction tube may conduct to at least apart of a site where the combining member is in contact with the gripping section. According to this aspect, while imparting a function of sucking the liquid to the gripping section, it is possible to impart a function of an electric knife to the gripping section even if an electrode is not separately provided.

(14) In the aspect described above, the liquid ejection device may further include a combining member coupled to the ejection tube and combined with the gripping section to attach the gripping section, and the distal end of the combining member and at least a part of a portion in contact with the gripping section may conduct to each other. According to this aspect, it is easy to avoid carbide, which occurs when the function of the electric knife is used, from adhering to the distal ends of the ejection tube and the suction tube.

(15) In the aspect described above, the gripping section may include a suction tube for sucking the liquid, at least a part of the connection channel may be a channel formed as an inner tube of a double tube, an outer tube of the double tube may be a suction channel, the attachment may be implemented by connecting the suction channel to an end portion of the suction tube, and any one of the ejection tube and the connection channel may project from an end portion of the outer tube of the double tube. According to this aspect, it is possible to separate the ejection tube and the suction tube by detaching the gripping section from the suction channel. Consequently, for example, it is possible to easily execute a change in the inner diameter of the suction tube.

(16) In the aspect described above, the liquid ejection device may further include a suction device configured to suck the liquid through the suction tube. According to this aspect, it is possible to suck the liquid.

(17) In the aspect described above, the pulsed-flow generating section may change the volume of the liquid chamber.

(18) In the aspect described above, the pulsed-flow generating section may generate air bubbles in the liquid chamber.

The invention can also be implemented as various aspects other than the aspects explained above. For example, the liquid ejection devices in the aspects can be implemented as medical apparatuses.

### BRIEF DESCRIPTION OF DRAWINGS

FIG. 1 is a configuration diagram of a liquid ejection device (a first embodiment).

FIG. 2 is a sectional view of a hand piece (the first embodiment).

FIG. 3 is a sectional view of the internal configuration of a pulsed-flow generating unit.

FIG. 4 is a diagram showing an example of a waveform of a drive voltage applied to a piezoelectric element.

FIG. 5 is a diagram showing a correspondence relation between the waveform of the drive voltage and a state of deformation of a diaphragm.

FIG. 6 is a configuration diagram of a liquid ejection device (a second embodiment).

FIG. 7 is a sectional view of a hand piece (the second embodiment).

FIG. 8 is a configuration diagram of a liquid ejection device (a third embodiment).

FIG. 9 is a sectional view of a hand piece (the third embodiment).

FIG. 10 is a configuration diagram of a liquid ejection device (a fourth embodiment).

FIG. 11 is a sectional view of a hand piece (the fourth embodiment).

FIG. 12 is a configuration diagram of a liquid ejection device (a fifth embodiment).

FIG. 13 is a sectional view of a hand piece (the fifth embodiment).

FIG. 14 is a configuration diagram of a liquid ejection device (a sixth embodiment).

FIG. 15 is a sectional view of a hand piece (the sixth embodiment).

FIG. 16 is a configuration diagram of a liquid ejection device (a seventh embodiment).

FIG. 17 is a sectional view of a hand piece and the periphery of the hand piece (the seventh embodiment).

FIG. 18 is a configuration diagram of a liquid ejection device (an eighth embodiment).

FIG. 19 is a sectional view of a hand piece and the periphery of the hand piece (the eighth embodiment).

FIG. 20 is a configuration diagram of a liquid ejection device (a ninth embodiment).

FIG. 21 is a sectional view of a hand piece and the periphery of the hand piece (the ninth embodiment).

FIG. 22 is a configuration diagram of a liquid ejection device (a tenth embodiment).

FIG. 23 is a sectional view of a hand piece and the periphery of the hand piece (the tenth embodiment).

FIG. 24 is a configuration diagram of a liquid ejection device (an eleventh embodiment).

FIG. 25 is a diagram showing a state in which a hand piece is separated (the eleventh embodiment).

FIG. 26 is a diagram showing a state in which the hand piece is attached (the eleventh embodiment).

FIG. 27 is a configuration diagram of a liquid ejection device (a twelfth embodiment).

### DESCRIPTION OF EMBODIMENTS

### First Embodiment

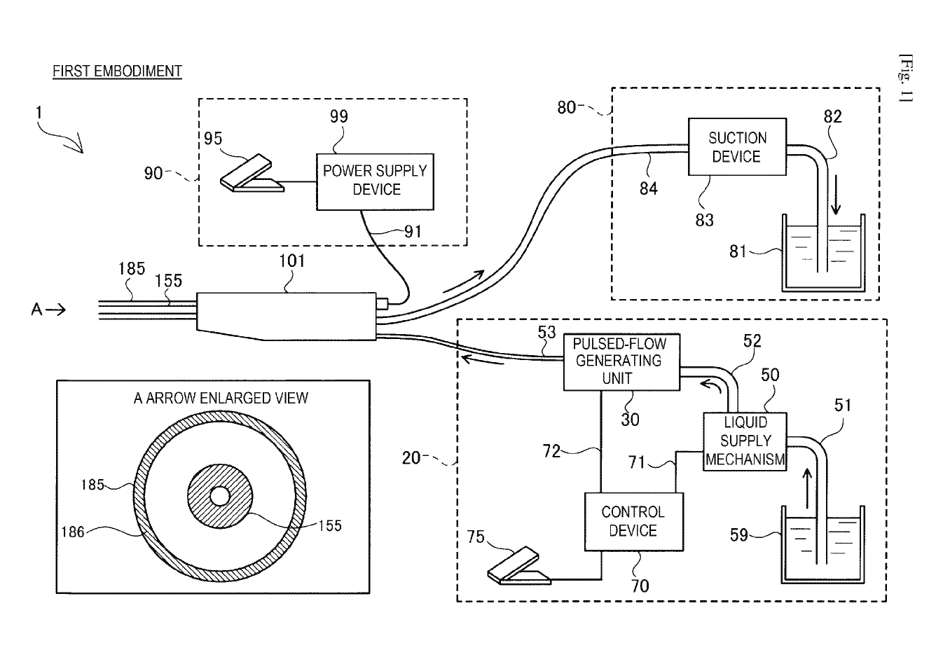

FIG. 1 shows the configuration of a liquid ejection device 1. The liquid ejection device 1 is a medical apparatus used in a medical institution. The liquid ejection device 1 includes a function of ejecting liquid to a diseased part in a pulse-like manner to thereby incise of excise the diseased part.

The liquid ejection device 1 includes a liquid supply unit 20, a liquid suction unit 80, a power supply unit 90, and a hand piece 101.

The liquid supply unit 20 includes a pulsed-flow generating unit 30, a liquid supply mechanism 50, a channel 51, a channel 52, a connection channel 53, a liquid container 59, a control device 70, a signal line 71, a signal line 72, and a footswitch 75.

The liquid supply mechanism 50 sucks, via the channel 51, liquid stored in the liquid container 59 and supplies the liquid to the pulsed-flow generating unit 30 via the channel 52. The liquid is saline. The pulsed-flow generating unit 30 generates a pulsed flow in the supplied liquid. The liquid, in which the pulsed-flow is generated, is supplied to the hand piece 101 via the connection channel 53. The connection channel 53 is formed of PEEK (registered trademark) resin. The PEEK resin is an insulator. The PEEK resin has flexibility and, on the other hand, has high rigidity.

The hand piece 101 is an instrument that a user of the liquid ejection device 1 holds with a hand and operates. The hand piece 101 includes an ejection tube 155 and a suction tube 185. The hand piece 101 is connected to a connection channel 53 and a suction channel 84 included in the liquid suction unit 80.

FIG. 2 shows a sectional view of the hand piece 101. The ejection tube 155 is made of metal. The ejection tube 155 is curved on the inside of the hand piece 101 and connected to the connection channel 53. The liquid supplied to the hand piece 101 via the connection channel 53 is ejected from the distal end of the ejection tube 155. Since the pulsed flow is generated in the liquid supplied to the hand piece 101, as explained above, the liquid is ejected in a pulse-like manner. Note that the pulse-like ejection in this application means that the liquid is ejected in a state in which a flow rate or flow velocity involve fluctuation. The pulse-like ejection is not limited to repetition of the ejection and a stop of the ejection of the liquid. That is, the pulse-like ejection includes various ejection forms such as a form in which ejection is completely interrupted between ejection and ejection and a form in which a flow with low pressure is present between ejections as well.

As shown in FIG. 1, the control device 70 controls the operation of the pulsed-flow generating unit 30 and the liquid supply mechanism 50. During start, the control device 70 always transmits, to the liquid supply mechanism 50 via the signal line 71, a signal for supplying the liquid. While the footswitch 75 is stepped on, the control device 70 transmits, to the pulsed-flow generating unit 30 via the signal line 72, a signal for generating a pulsed flow.

As shown in FIG. 2, the suction tube 185 is formed in a double tube structure in which the ejection tube 155 is an inner tube and the suction tube 185 is an outer tube. The suction tube 185 is made of metal. The ejection tube 155 pierces through a hole HL provided in the suction tube 185. The liquid suction unit 80 sucks, via the suction tube 185 and the suction channel 84, the liquid in the vicinity of an end portion 186 (FIG. 2) of the suction tube 185.

As shown in FIG. 1, the liquid suction unit 80 includes a drain container 81, a channel 82, a suction device 83, and a suction channel 84. The suction channel 84 is an insulator formed of resin. The suction channel 84 is connected to the suction tube 185 on the inside of the hand piece 101. The suction device 83 sucks the inside of a tube of the suction channel 84 to thereby suck the liquid in the vicinity of the distal end of the suction tube 185 and a solid body caused by incision. The liquid and the solid body sucked by the suction device 83 are discarded to the drain container 81 via the channel 82.

As shown in FIG. 1, the power supply unit 90 includes a cable 91, a footswitch 95, and a power supply device 99. While the footswitch 95 is stepped on, the power supply device 99 supplies high-frequency power to the hand piece 101 via the cable 91.

As shown in FIG. 2, the hand piece 101 includes a connection terminal 193. The connection terminal 193 is a terminal detachably attachable to a connection terminal 92 provided at an end portion of the cable 91. The connection terminal 193 conducts to the suction tube 185 via a wire 161. Therefore, when the power supply device 99 supplies the high-frequency power, the end portion 186 of the suction tube 185 functions as an electrode of an electric knife of a mono-polar type. Note that a well-known counter electrode plate (not shown in the figure) is used in order to cause the end portion 186 to function as the electric knife of the mono-polar type in this way.

The connection channel 53 and the suction channel 84 are formed of the insulator as explained above in order to insulate the high-frequency power. The liquid flowing on the insides of the connection channel 53 and the suction channel 84 is a conductor because the liquid is saline. However, the impedance of the saline generally interrupts the high-frequency power. Therefore, the high-frequency power hardly affects the pulsed-flow generating unit 30 and the suction device 83.

As shown in FIG. 2, the hand piece 101 includes vibration absorbing members 121 and 131. The vibration absorbing members 121 and 131 are formed of an elastic body such as rubber. The vibration absorbing member 121 absorbs vibration of the connection channel 53. The vibration absorbing member 131 absorbs vibration of the ejection tube 155. Vibration transmitted from the pulsed-flow generating unit 30 via the connection channel 53 is suppressed by the vibration absorbing members 121 and 131. Vibration transmitted to the hand, which is gripping the hand piece 101, is reduced.

FIG. 3 is a sectional view of the internal configuration of the pulsed-flow generating unit 30. The pulsed-flow generating unit 30 includes a pulsed-flow generating section 31 and a liquid chamber 42. The pulsed-flow generating section 31 includes a diaphragm 32 and a piezoelectric element 33.

The liquid chamber 42 is a space between a first case 34 and the diaphragm 32. The liquid chamber 42 forms a channel between the channel 52 and the connection channel 53. The diaphragm 32 is a disk-like metal thin plate. The diaphragm 32 is fixed with the outer circumferential portion thereof sandwiched between the first case 34 and a second case 36.

The piezoelectric element 33 is an actuator that operates with a drive voltage applied from the control device 70. The piezoelectric element 33 varies the volume of the liquid chamber 42 formed between the diaphragm 32 and the first case 34 to thereby vary the pressure of the liquid in the liquid chamber 42. The piezoelectric element 33 is a stacked piezoelectric element. One end of the piezoelectric element 33 is fixed to the diaphragm 32 and the other end thereof is fixed to a third case 38.

When the drive voltage applied to the piezoelectric element 33 increases, the piezoelectric element 33 expands. The diaphragm 32 is pushed by the piezoelectric element 33 to bend to the liquid chamber 42 side. When the diaphragm 32 bends to the liquid chamber 42 side, the volume of the liquid chamber 42 decreases. The liquid in the liquid chamber 42 is pushed out from the liquid chamber 42 to the connection channel 53.

On the other hand, when the drive voltage applied to the piezoelectric element 33 decreases, the piezoelectric element 33 contracts. The volume of the liquid chamber 42 increases. The liquid flows into the liquid chamber 42 from the channel 52.

The drive voltage applied to the piezoelectric element 33 from the control device 70 repeats ON (a maximum voltage) and OFF (0 V) at a predetermined frequency (e.g., 400 Hz). Therefore, the increase and the decrease of the volume of the liquid chamber 42 are repeated. A pulsed flow is generated in the liquid.

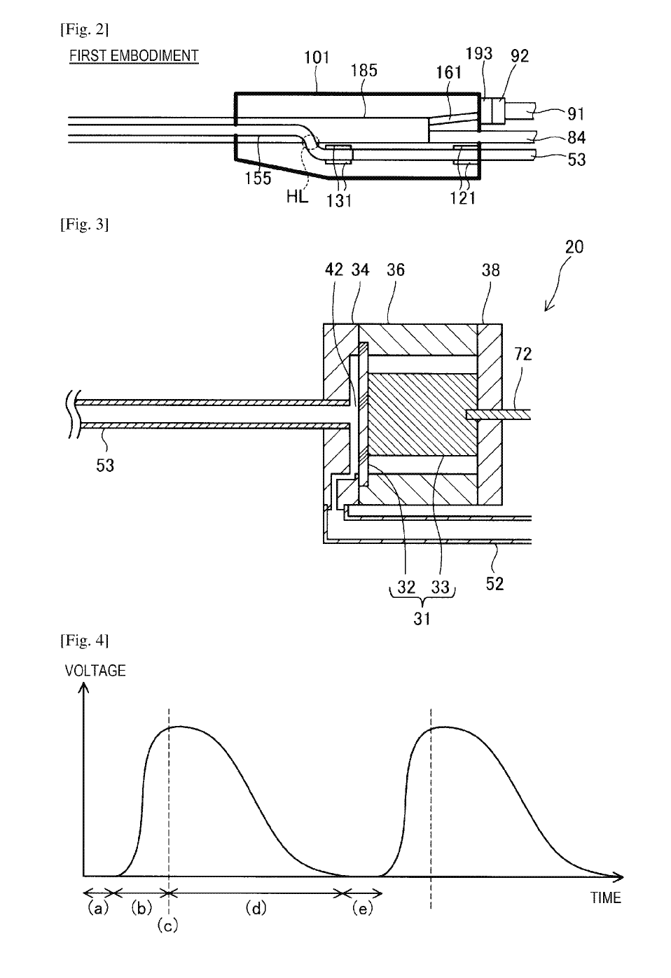

FIG. 4 shows an example of a waveform of the drive voltage applied to the piezoelectric element 33. In FIG. 4, the abscissa indicates time and the ordinate indicates the drive voltage. One cycle of the waveform of the drive voltage is formed by a rising period (b) in which the voltage increases, time (c) when the voltage is the maximum, a falling period (d) in which the voltage decreases, and quiescent periods (a) and (e) in which the voltage is not applied.

A waveform in the rising period of the drive voltage is a waveform for a half cycle of a sine waveform that is offset in a positive voltage direction and phase-shifted by −90 degrees. A waveform in the falling period of the drive voltage is a waveform for the half cycle of the sine waveform that is offset in the positive voltage direction and phase-shifted by +90 degrees. The cycle of the sine waveform in the falling period is larger than the cycle of the sine waveform in the rising period.

When the magnitude of the drive voltage is changed, the maximum of the waveform shown in FIG. 4 is changed. When the frequency of the drive voltage is changed, the waveforms in the rising period and the falling period are not changed and the lengths of the quiescent periods are changed.

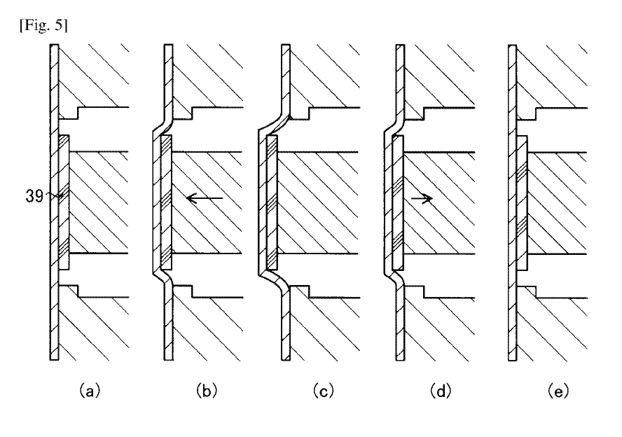

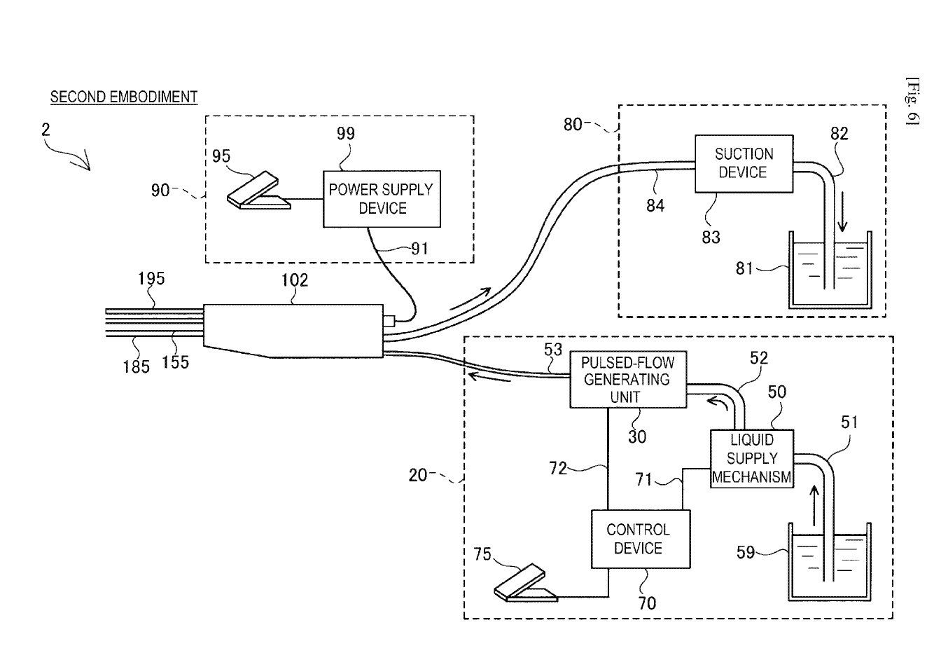

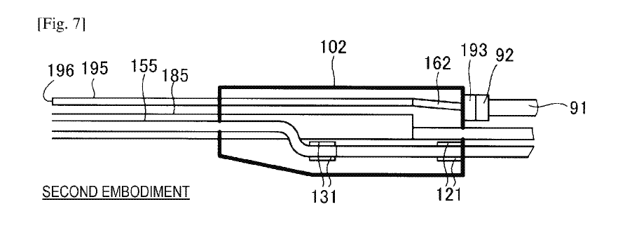

FIG. 5 shows a correspondence relation between the waveform of the drive voltage and a state of deformation of the diaphragm 32. Note that, in FIG. 5, a reinforcing member 39 is provided between the piezoelectric element 33 and the diaphragm 32. In the quiescent period (a), since the drive voltage is not applied, the piezoelectric element 33 does not expand and the diaphragm 32 does not bend. In the rising period (b), since the drive voltage increases, the piezoelectric element 33 expands, the diaphragm 32 bends to the liquid chamber 42 side, and the volume of the liquid chamber 42 decreases.

At the time (c), since the drive voltage is the maximum, the length of the piezoelectric element 33 is also the maximum and the volume of the liquid chamber 42 is the minimum. In the falling period (d), since the drive voltage decreases, the piezoelectric element 33 starts to return to the original size and the volume of the liquid chamber 42 starts to return to the original size. In the quiescent period (e), since the drive voltage is not applied, the piezoelectric element 33 returns to the original size and the volume of the liquid chamber 42 returns to the original size. The series of operation indicated by (a) to (e) is repeated, whereby a pulsed-flow is generated in the liquid in the liquid chamber 42.

According to the first embodiment, it is possible to obtain at least effects explained below.

(1A) The hand piece 101 is lighter than in the past. This is because the pulsed-flow generating unit 30 is disposed on the outside of the hand piece 101.

(1B) It is possible to impart the function of the electric knife to the hand piece 101.

(1C) Even if the function of the electric knife is imparted to the hand piece 101, since the pulsed-flow generating unit 30 is disposed on the outside of the hand piece 101, it is possible to implement electric protection without complicating the configuration in the hand piece 101. Consequently, an increase in the weight of the hand piece 101 is suppressed.

(1D) Even if the function of the electric knife is imparted to the hand piece 101, since the end portion 186 of the suction tube 185 is used as the electrode, it is unnecessary to separately provide an electrode member. Consequently, an increase in the weight of the hand piece 101 is suppressed.

(1E) When the function of the electric knife is not used, the hand piece 101 is further reduced in weight by detaching the cable 91.

(1F) It is possible to suppress vibration from the connection channel 53 with the vibration absorbing members 121 and 131.

(1G) Since the suction tube 185 has a linear shape, clogging in the tube is suppressed and manufacturing costs are reduced.

(1H) It is possible to separately replace and clean the pulsed-flow generating unit 30 and the hand piece 101.

### Second Embodiment

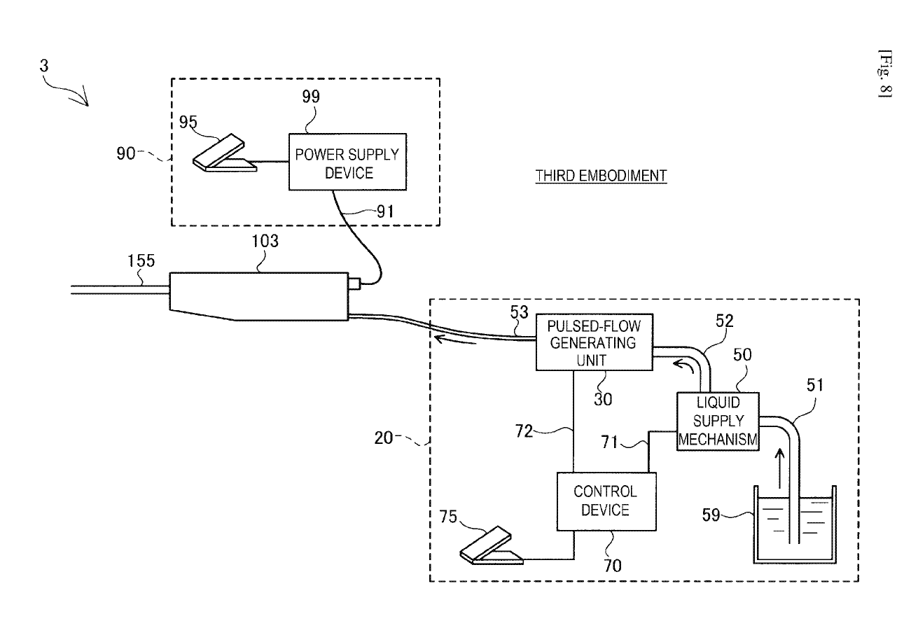

FIG. 6 shows the configuration of a liquid ejection device 2. The liquid ejection device 2 is different from the liquid ejection device 1 in that the liquid ejection device 2 includes a hand piece 102 instead of the hand piece 101. The other components are the same as the components of the liquid ejection device 1.

FIG. 7 shows a sectional view of the hand piece 102. Unlike the hand piece 101, the hand piece 102 includes an electrode member 195. The electrode member 195 conducts to the connected cable 91 via a wire 162. In the hand piece 102, instead of the suction tube 185, an end portion 196 of the electrode member 195 functions as an electrode of an electric knife.

According to the second embodiment, even if the function of the electric knife is used, adhesion of carbide to the end portion 186 of the suction tube 185 is suppressed. In addition, since the electrode of the electric knife can be freely disposed, it is easy to perform design corresponding to a use or preference of a surgeon.

### Third Embodiment

FIG. 8 shows the configuration of a liquid ejection device 3. The liquid ejection device 3 is configured by connecting a hand piece 103 to the liquid supply unit 20 and the power supply unit 90. On the other hand, unlike the liquid ejection device 1, the liquid ejection device 3 does not include the liquid suction unit 80 and the suction tube 185.

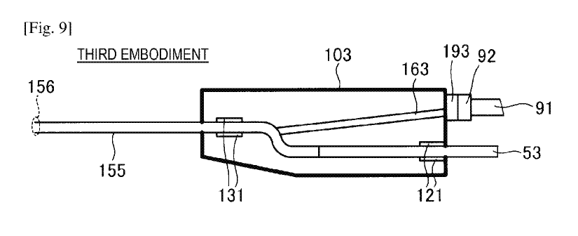

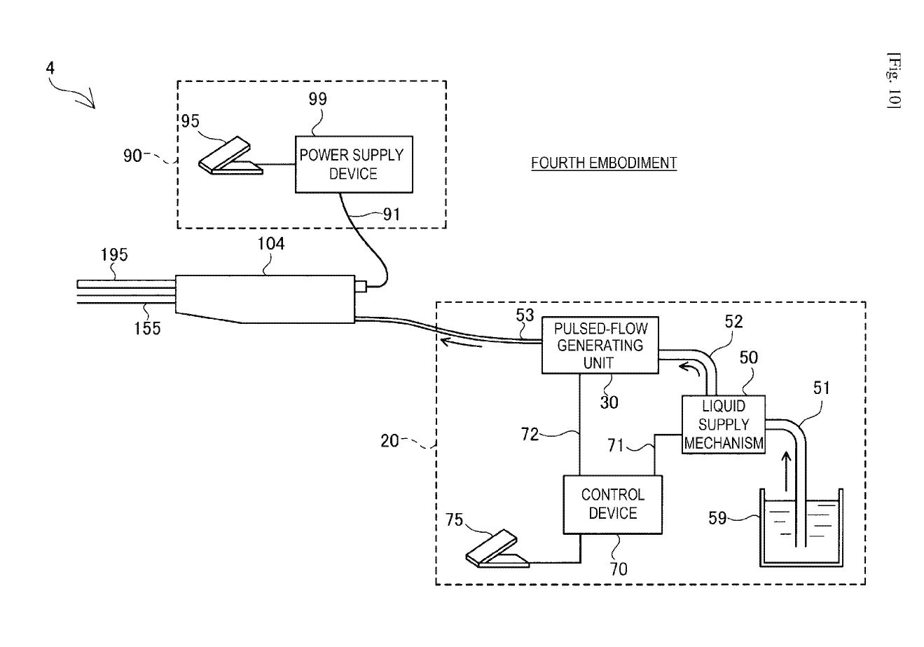

FIG. 9 shows a sectional view of the hand piece 103. The hand piece 103 includes a wire 163. The wire 163 causes the connection terminal 193 and the ejection tube 155 to conduct to each other. In the hand piece 103, an end portion 156 of the ejection tube 155 functions as an electrode of an electric knife.

In the hand piece 103, compared with the hand piece 101, the vibration absorbing member 131 is disposed closer to the end portion 156 of the ejection tube 155. This disposition makes use of the fact that the suction tube 185 is not provided. With this disposition, vibration is further reduced.

According to the third embodiment, since the hand piece 103 does not include the suction tube 185, the hand piece 103 is further reduced in weight. In addition, the hand piece 103 is easy to use because an ejection position and the electrode of the electric knife are the same.

### Fourth Embodiment

FIG. 10 shows the configuration of a liquid ejection device 4. The liquid ejection device 4 is different from the liquid ejection device 3 in that the liquid ejection device 4 includes a hand piece 104 instead of the hand piece 103. The other components are the same as the components of the liquid ejection device 3.

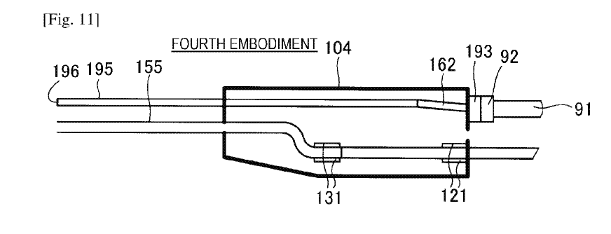

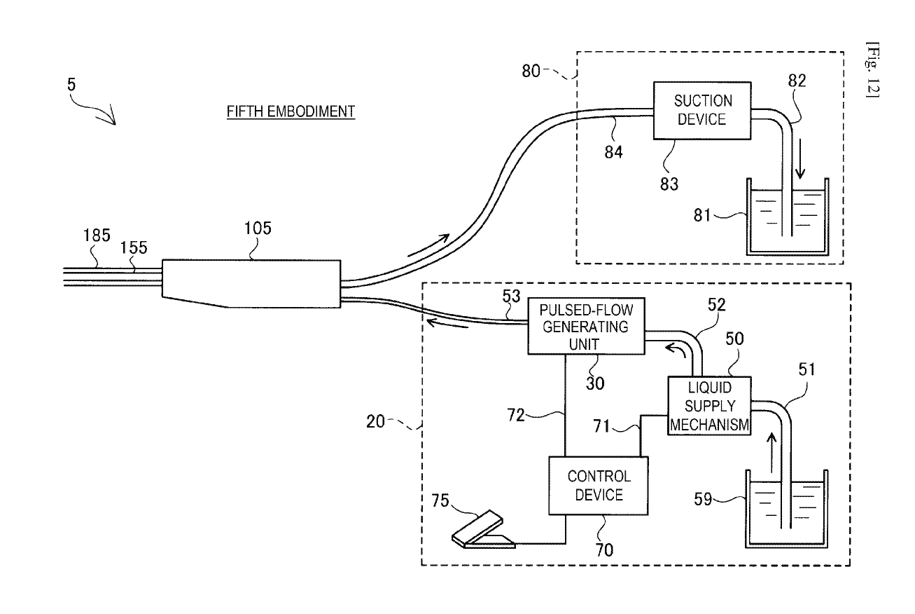

FIG. 11 shows a sectional view of the hand piece 104. Like the hand piece 103, the hand piece 104 includes the ejection tube 155. Like the hand piece 102, the hand piece 104 has a configuration in which high-frequency power is supplied to the electrode member 195.

According to the fourth embodiment, even if a function of an electric knife is used, it is possible to suppress adhesion of carbide to the end portion 156 of the ejection tube 155.

### Fifth Embodiment

FIG. 12 shows the configuration of a liquid ejection device 5. The liquid ejection device 5 is configured by connecting a hand piece 105 to the liquid supply unit 20 and the liquid suction unit 80. On the other hand, unlike the liquid ejection device 1, the liquid ejection device 5 does not include the power supply unit 90.

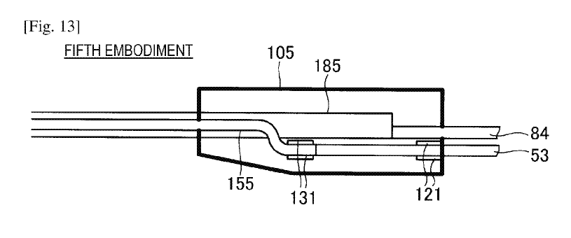

FIG. 13 shows a sectional view of the hand piece 105. Unlike the hand piece 101, since the hand piece 105 does not receive supply of high-frequency power, the hand piece 105 does not include the wire 161 and the connection terminal 193.

According to the fifth embodiment, since the hand piece 105 does not include the wire 161 and the connection terminal 193, the hand piece 105 is further reduced in weight.

### Sixth Embodiment

FIG. 14 shows the configuration of a liquid ejection device 6. The liquid ejection device 6 is configured by connecting a hand piece 106 to the liquid supply unit 20. On the other hand, unlike the liquid ejection device 1, the liquid ejection device 6 does not include the liquid suction unit 80 and the power supply unit 90.

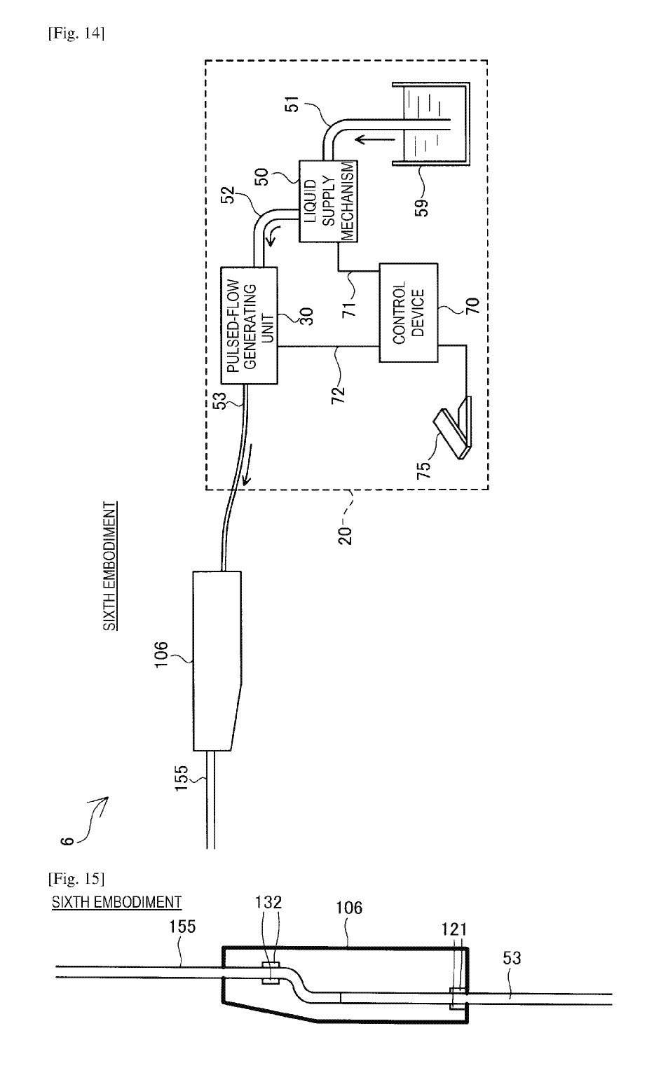

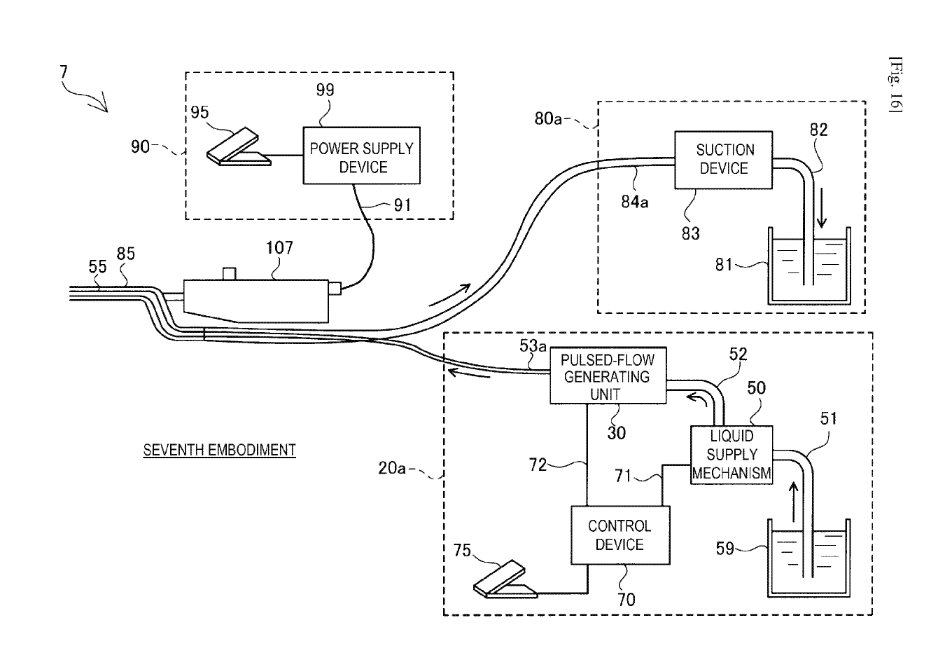

FIG. 15 shows a sectional view of the hand piece 106. Unlike the hand piece 101, since the hand piece 106 does not receive supply of high-frequency power, the hand piece 106 does not include the wire 161 and the connection terminal 193. Unlike the hand piece 101, since the hand piece 106 is not sucked by the liquid suction unit 80, the hand piece 106 does not include the suction tube 185.

According to the sixth embodiment, since the hand piece 106 does not include the suction tube 185, the wire 161, and the connection terminal 193, the hand piece 106 is further reduced in weight.

### Seventh Embodiment

FIG. 16 shows the configuration of a liquid ejection device 7. The liquid ejection device 7 includes a liquid supply unit 20a, an ejection tube 55, a liquid suction unit 80a, a suction tube 85, the power supply unit 90, and a hand piece 107.

The liquid supply unit 20a is different from the liquid supply unit 20 in the first embodiment in that the liquid supply unit 20a includes a connection channel 53a instead of the connection channel 53. The other components are the same as the components of the liquid supply unit 20. The liquid suction unit 80a is different from the liquid suction unit 80 in the first embodiment in that the liquid suction unit 80a includes a suction channel 84a instead of the suction channel 84. The other components are the same as the components of the liquid suction unit 80.

As shown in FIG. 16, the entire connection channel 53a and the entire suction channel 84a are disposed on the outside of the hand piece 107. The ejection tube 55 is made of metal. The ejection tube 55 is connected to the connection channel 53a on the outside of the hand piece 107. The suction tube 85 is made of metal. The suction tube 85 is connected to the suction channel 84a on the outside of the hand piece 107. The hand piece 107 is connected to the suction tube 85.

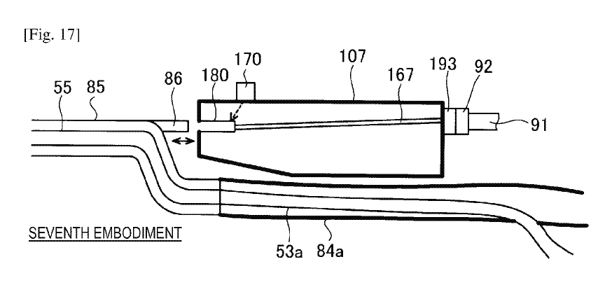

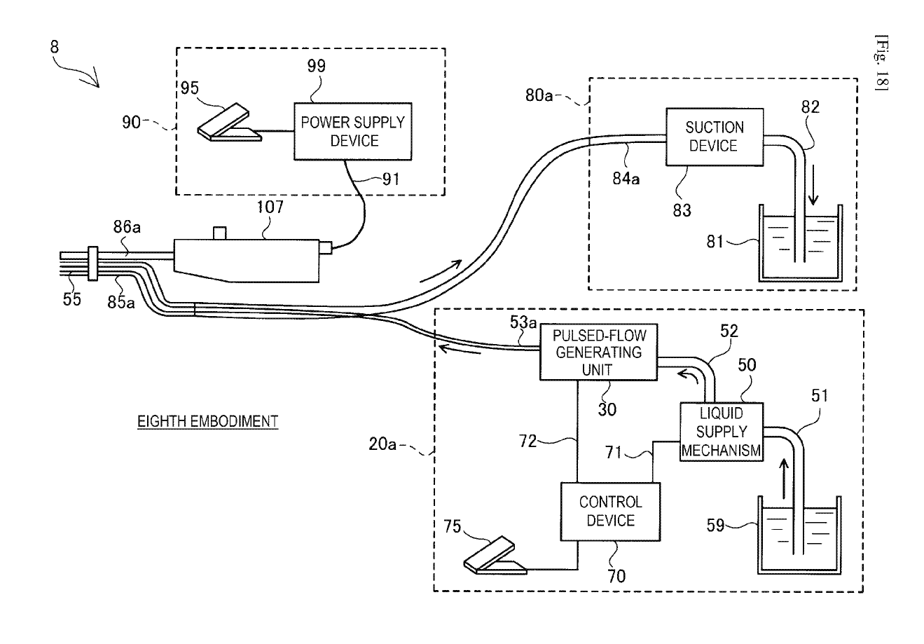

FIG. 17 shows a sectional view of the hand piece 107 and the periphery of the hand piece 107. FIG. 17 shows a state in which the hand piece 107 is not connected to the suction tube 85. A hole 180 is provided in the hand piece 107. A protrusion 86 functioning as a combining member is provided in the suction tube 85. The protrusion 86 is pushed into the hole 180, whereby the hand piece 107 is attached to the suction tube 85. If the protrusion 86 is pulled out from the hole 180, the hand piece 107 is separated from the suction tube 85. In this way, the hand piece 107 has a configuration detachably attachable to the suction tube 85.

The hand piece 107 includes a wire 167. The wire 167 causes the connection terminal 193 and the vicinity of the bottom section of the hole 180 to conduct to each other. The protrusion 86 is made of metal. Therefore, when the protrusion 86 is pushed into the depth of the hole 180, the connection terminal 193 and the suction tube 85 conduct to each other. If the connection terminal 92 is connected to the connection terminal 193, an end portion of the suction tube 85 receives supply of high-frequency power from the power supply unit 90 and functions as an electrode of an electric knife. The supply of the high-frequency power from the power supply unit 90 is executed while a switch 170 provided in the hand piece 107 is pushed.

According to the seventh embodiment, it is possible to obtain at least effects explained below.

(7A) A substantial increase in the weight of the hand piece 107 is avoided. This is because it is possible to design the hand piece 107 not to include the pulsed-flow generating unit 30.

(7B) It is possible to replace and clean the ejection tube 55 and the suction tube 85 separately from the hand piece 107. For example, it is possible to replace the connection channel 53a, the ejection tube 55, the suction channel 84a, and the suction tube 85 in every surgical operation and clean the hand piece 107 in every surgical operation.

(7C) Since a handle of an existing electric knife can be used as the hand piece 107, it is possible to inexpensively prepare the hand piece 107.

### Eighth Embodiment

FIG. 18 shows the configuration of a liquid ejection device 8. The liquid ejection device 8 is different from the liquid ejection device 7 in that the liquid ejection device 8 includes a suction tube 85a instead of the suction tube 85 and includes an electrode member 86a instead of the protrusion 86. The other components are the same as the components of the liquid ejection device 7.

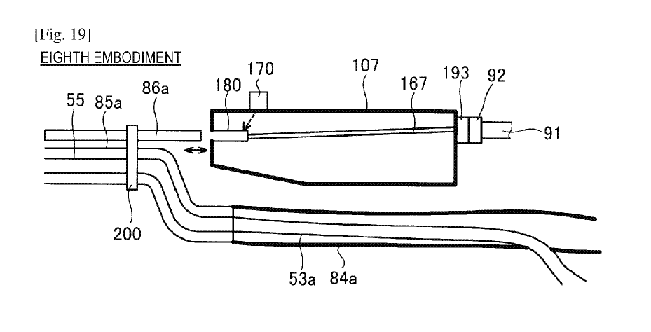

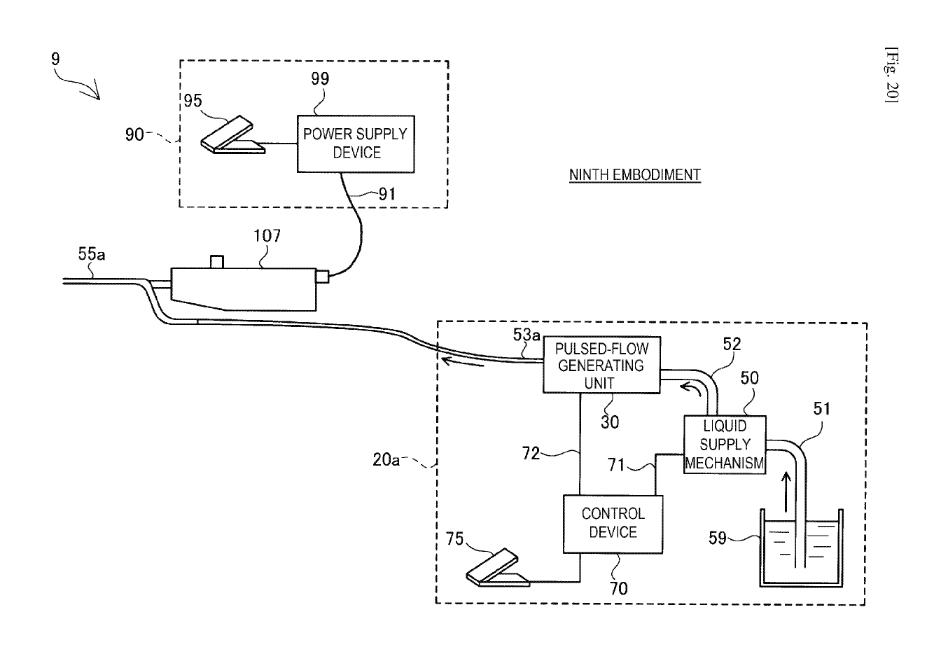

FIG. 19 shows a sectional view of the hand piece 107 and the periphery of the hand piece 107 in the eighth embodiment. The electrode member 86a is coupled to the suction tube 85a by an insulative coupling member 200. Like the protrusion 86, the electrode member 86a is a member made of metal and pushed into the hole 180. That is, the electrode member 86a is a member for connecting the hand piece 107. An end portion of the electrode member 86a functions as an electrode of an electric knife. According to the eighth embodiment, adhesion of carbide to an end portion of the suction tube 85a is suppressed.

### Ninth Embodiment

FIG. 20 shows the configuration of a liquid ejection device 9. Like the liquid ejection device 7, the liquid ejection device 9 includes the liquid supply unit 20a, the power supply unit 90, and the hand piece 107. On the other hand, the liquid ejection device 9 does not include the liquid suction unit 80a and the suction tube 85 and includes an ejection tube 55a instead of the ejection tube 55.

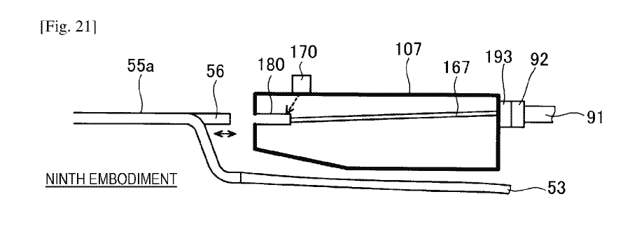

FIG. 21 shows a sectional view of the hand piece 107 and the periphery of the hand piece 107 in the ninth embodiment. The ejection tube 55a includes a protrusion 56. Like the protrusion 86 in the seventh embodiment, the protrusion 56 includes a function of attaching the hand piece 107 and a function of causing the ejection tube 55a and the cable 91 to conduct to each other. In the ninth embodiment, an end portion of the ejection tube 55a functions as an electrode of an electric knife.

According to the ninth embodiment, since the suction tube 85 is not coupled to the hand piece 107, it is easier to operate the hand piece 107.

### Tenth Embodiment

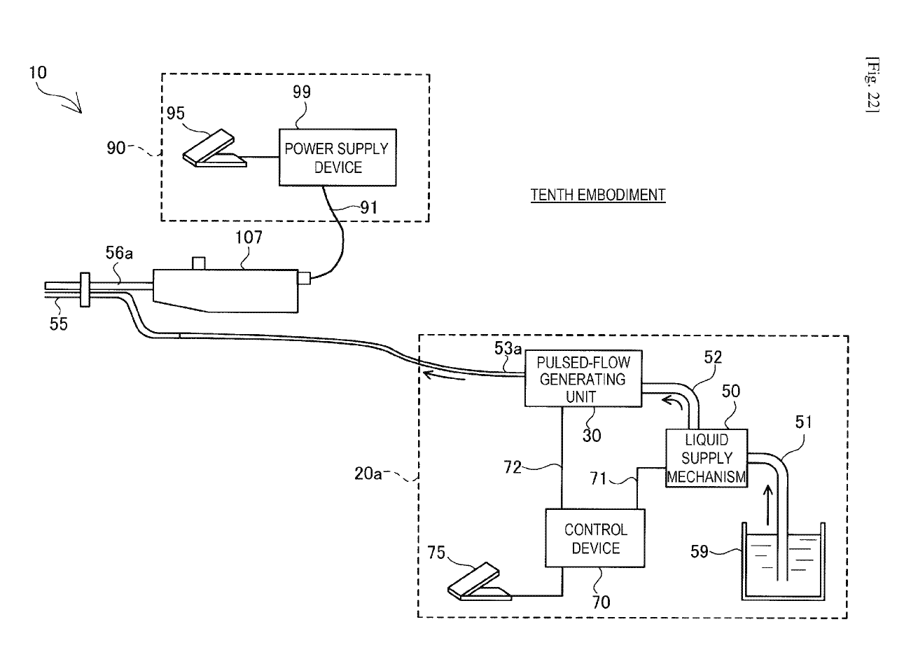

FIG. 22 shows the configuration of a liquid ejection device 10. The liquid ejection device 10 is different from the liquid ejection device 9 in that the liquid ejection device 10 includes the ejection tube 55 instead of the ejection tube 55a and includes an electrode member 56a instead of the protrusion 56. The other components are the same as the components of the liquid ejection device 9.

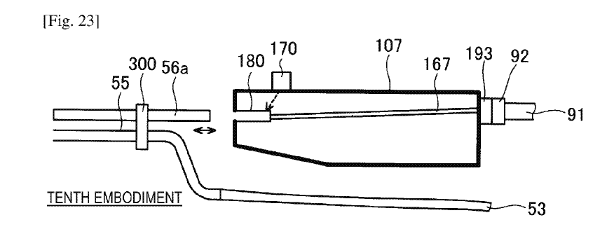

FIG. 23 shows a sectional view of the hand piece 107 and the periphery of the hand piece 107 in the tenth embodiment. The electrode member 56a is coupled to the ejection tube 55 by an insulative coupling member 300. Like the electrode member 86a in the eighth embodiment, the electrode member 56a includes a function of attaching the hand piece 107 to the ejection tube and a function of an electrode of an electric knife. According to the tenth embodiment, adhesion of carbide to the end portion of the ejection tube 55 is suppressed.

### Eleventh Embodiment

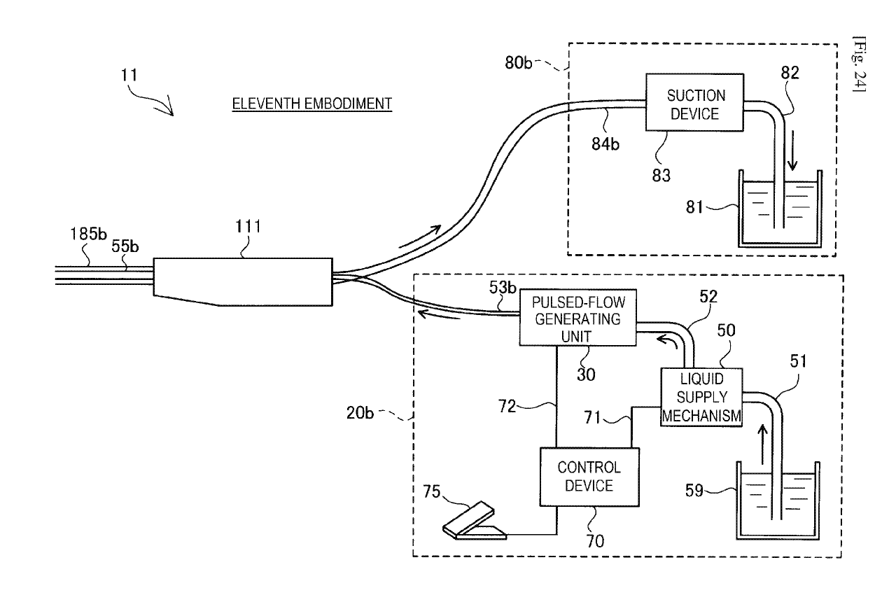

FIG. 24 shows the configuration of a liquid ejection device 11. The liquid ejection device 11 includes a liquid supply unit 20b, a liquid suction unit 80b, and a hand piece 111.

The liquid supply unit 20b is different from the liquid supply unit 20 in the first embodiment in that the liquid supply unit 20b includes a connection channel 53b instead of the connection channel 53. The other components are the same as the components of the liquid supply unit 20. The liquid suction unit 80b is different from the liquid suction unit 80 in the first embodiment in that the liquid suction unit 80b includes a suction channel 84b instead of the suction channel 84. The other components are the same as the components of the liquid suction unit 80.

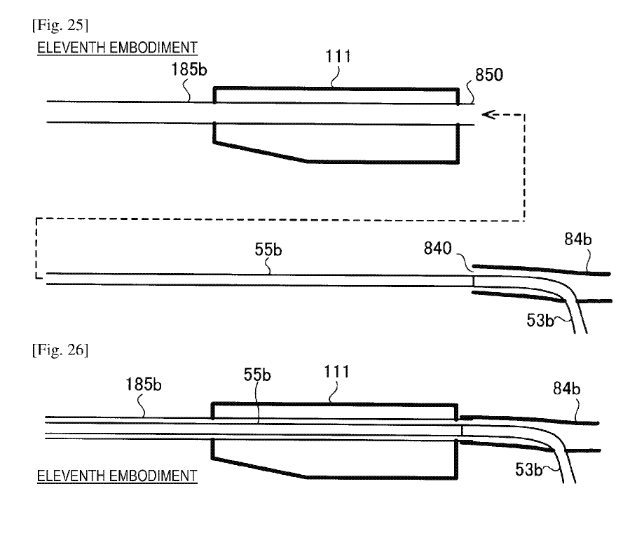

FIG. 25 shows a state in which the hand piece 111 is separated from the suction channel 84b. An ejection tube 55b is connected to the connection channel 53b. The ejection tube 55b projects from an opening 840 of the suction channel 84b. As shown in FIG. 25, a double tube is formed in which at least a part of the connection channel 53b is an inner tube and at least apart of the suction channel 84b is an outer tube. Therefore, even if the hand piece 111 and the suction channel 84b are separated, the connection channel 53b and the suction channel 84b are integrated. It is easy to handle the connection channel 53b and the suction channel 84b.

A suction tube 185b pierces through the hand piece 111 and forms an end portion 850 located on the opposite side of a suction port. When the hand piece 111 is connected from a separated state, the ejection tube 55b is inserted into the suction tube 185b from the end portion 850.

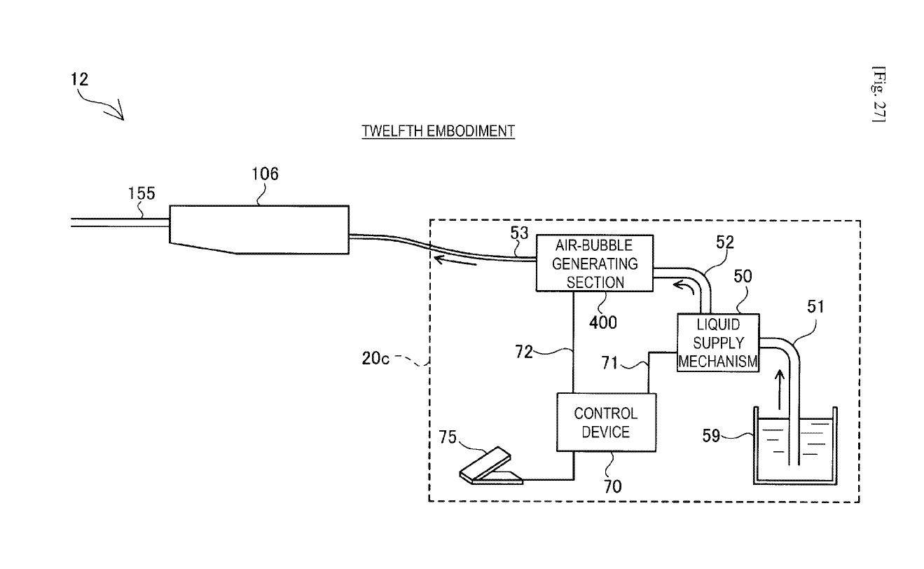

FIG. 26 shows a state in which the hand piece 111 is attached to the suction channel 84b. When the ejection tube 55b is inserted into the suction tube 185b, the end portion 850 of the suction tube 185b can be inserted into the opening 840 of the connection channel 53b and attached. When the end portion 850 is attached in this way, the ejection tube 55b can be operated by the hand piece 111.

According to the eleventh embodiment, it is possible to obtain at least effects explained below.

(11A) A great increase in the weight of the hand piece 111 is avoided. This is because the pulsed-flow generating unit 30 is disposed on the outside of the hand piece 111.

(11B) Since the ejection tube 55b and the suction tube 185b have linear shapes, clogging in the tubes is suppressed and manufacturing costs are reduced.

(11C) It is possible to replace only the hand piece 111 and the suction tube 185b. Consequently, if a plurality of the hand pieces 111 including the suction tubes 185b respectively having different inner diameters are prepared, it is possible to easily change the inner diameter of the suction tube 185b.

(11D) Since an existing suction tube can be used as the hand piece 111, it is possible to inexpensively prepare the hand piece 111. The “existing suction tube” is known as a surgical instrument in which members equivalent to the hand piece 111 and the suction tube 185b are integrally molded.

### Twelfth Embodiment

FIG. 27 shows the configuration of a liquid ejection device 12. The liquid ejection device 12 is different from the liquid ejection device 6 in that the liquid ejection device 12 includes an air-bubble generating section 400 instead of the pulsed-flow generating unit 30. The other components are the same as the components of the liquid ejection device 6.

The air-bubble generating section 400 intermittently generates air bubbles in a liquid chamber incorporated therein to thereby generate a pulsed flow in liquid in the liquid chamber. The air-bubble generating section 400 generates air bubbles in the liquid chamber using an optical maser. According to the twelfth embodiment, it is possible to generate a pulsed flow using the optical maser.

The invention is not limited to the embodiments, the examples, and the modifications of this specification and can be implemented as various configurations without departing from the spirit of the invention. For example, the technical features in the embodiments, the examples, and the modifications corresponding to the technical features in the aspects described in the summary of the invention can be interchanged or combined as appropriate in order to solve apart or all of the problems explained above or in order to attain a part of all of the effects explained above. If the technical features are not explained as essential features in this specification, the technical features can be deleted as appropriate. For example, technical features explained below are illustrated.

When the gripping section includes the suction tube, a suction adjustment hole may be provided in the gripping section. The suction adjustment hole is a hole that opens the suction tube to the atmosphere separately from the distal end of the suction tube. By closing the suction adjustment hole with a finger and opening the suction adjustment hole, it is possible to adjust a suction force at the end portion of the suction tube.

The cable for supplying high-frequency power may be connected to the hand piece not via the connection terminal. For example, the cable may be fixed to the hand piece.

The gripping section does not have to be the hand piece and may be, for example, a flexible tube used in an endoscope. The flexible tube is a hollow member that can be bent by remote operation. Insertion of the ejection tube or insertion of the ejection tube and the suction tube into the inside of the flexible tube is included in “attachment of the gripping section and the ejection tube” in this application.

The liquid ejection device explained as being used using the detachably configured hand piece 107 may be used without using the hand piece 107. For example, the user may directly grip the ejection tube or the suction tube.

When the detachably configured hand piece 107 is used, the connection channel 53a and the suction channel 84a may be fastened to the hand piece 107 using a fastening band or the like.

The air-bubble generating section may generate air bubbles using a heater.

The materials of the ejection tube, the suction tube, the connection channel, the suction channel, and the like may be changed as appropriate.

Only one vibration absorbing member may be provided, three or more vibration absorbing member may be provided, or no vibration absorbing member may be provided.

The liquid to be ejected may be pure water, chemical, or the like.

The liquid ejection device may be used in an apparatus other than the medical apparatus.

For example, the liquid ejection device may be used in a cleaning apparatus that removes stain with ejected liquid.

The liquid ejection device may be used in a drawing apparatus that draws lines and the like with ejected liquid.

### REFERENCE SIGNS LIST

1 Liquid ejection device
2 Liquid ejection device
3 Liquid ejection device
4 Liquid ejection device
5 Liquid ejection device
6 Liquid ejection device
7 Liquid ejection device
8 Liquid ejection device
9 Liquid ejection device
10 Liquid ejection device
11 Liquid ejection device
12 Liquid ejection device
20 Liquid supply unit
20a Liquid supply unit
20b Liquid supply unit
30 Pulsed-flow generating unit
31 Pulsed-flow generating section
32 Diaphragm
33 Piezoelectric element
34 First case
36 Second case
38 Third case
39 Reinforcing member
42 Liquid chamber
50 Liquid supply mechanism
51 Channel
52 Channel
53 Connection channel
53a Connection channel
53b Connection channel
55 Ejection tube
55a Ejection tube
55b Ejection tube
56 Protrusion
56a Electrode member
59 Liquid container
70 Control device
71 Signal line
72 Signal line
75 Footswitch
80 Liquid suction unit
80a Liquid suction unit
80b Liquid suction unit
81 Drain container
82 Channel
83 Suction device
84 Suction channel
84a Suction channel
84b Suction channel
85 Suction tube
85a Suction tube
86 Protrusion
86a Electrode member
90 Power supply unit
91 Cable
92 Connection terminal
95 Footswitch
99 Power supply device
101 Hand piece
102 Hand piece
103 Hand piece
104 Hand piece
105 Hand piece
106 Hand piece
107 Hand piece
111 Hand piece
121 Vibration absorbing member
131 Vibration absorbing member
155 Ejection tube
156 End portion
161 Wire
162 Wire
163 Wire
167 Wire
170 Switch
180 Hole
185 Suction tube
185b Suction tube
186 End portion
193 Connection terminal
195 Electrode member
196 End portion
200 Coupling member
300 Coupling member
400 Air-bubble generating section
850 End portion
HL Hole

1. A liquid ejection device comprising:
an ejection tube for ejecting liquid;
a gripping section attached to the ejection tube and gripped by a user;
a liquid chamber provided on an outside of the gripping section;
a connection channel configured to connect the liquid chamber and the ejection tube; and
a pulsed-flow generating section provided on an outside of the gripping section and configured to generate a pulsed flow in the liquid in the liquid chamber.

2. The liquid ejection device according to claim 1, further comprising a power supply device configured to supply high-frequency power to an electrode provided in the gripping section.

3. The liquid ejection device according to claim 1, wherein
the gripping section includes a connection terminal to which a cable is detachably attachable, and
the connection terminal and an electrode provided in the gripping section conduct to each other.

4. The liquid ejection device according to claim 3, wherein the electrode is a distal end of the ejection tube.

5. The liquid ejection device according to claim 3, wherein
the gripping section includes a suction tube for sucking the liquid, and
the electrode is a distal end of the suction tube.

6. The liquid ejection device according to claim 3, wherein the gripping section further includes a member that conducts to the connection terminal and forms the electrode.

7. The liquid ejection device according to claim 1, wherein the gripping section and the ejection tube are detachably attachable.

8. The liquid ejection device according to claim 1, wherein
the gripping section includes a suction tube for sucking the liquid.

9. The liquid ejection device according to claim 8, wherein
at least a part of the connection channel is a channel formed as an inner tube of a double tube,
an outer tube of the double tube is at least a part of a suction channel detachably attached to an end portion of the suction tube, and
any one of the ejection tube and the connection channel projects from an end portion of the outer tube of the double tube.

10. The liquid ejection device according to claim 1, wherein
the gripping section includes a vibration absorbing member that absorbs vibration from the connection channel.

11. A liquid ejection device comprising:
an ejection tube for ejecting liquid, a gripping section gripped by a user being attachable to the ejection tube;
a connection channel configured to connect a liquid chamber and the ejection tube; and
a pulsed-flow generating section configured to generate a pulsed flow in the liquid in the liquid chamber.

12. The liquid ejection device according to claim 11, wherein
the ejection tube includes a combining member for the attachment, and
the distal end of the ejection tube conducts to at least a part of a site where the combining member is in contact with the gripping section.

13. The liquid ejection device according to claim 11, further comprising a suction tube coupled to the ejection tube to suck liquid, wherein
the attachment is implemented via the suction tube,
the suction tube includes a combining member for the attachment, and
a distal end of the suction tube conducts to at least a part of a site where the combining member is in contact with the gripping section.

14. The liquid ejection device according to claim 11, further comprising a combining member coupled to the ejection tube and combined with the gripping section to attach the gripping section, wherein
a distal end of the combining member and at least a part of a portion in contact with the gripping section conduct to each other.

15. The liquid ejection device according to claim 11, wherein
the gripping section includes a suction tube for sucking the liquid,
at least a part of the connection channel is a channel formed as an inner tube of a double tube,
an outer tube of the double tube is a suction channel,
the attachment is implemented by connecting the suction channel to an end portion of the suction tube, and
any one of the ejection tube and the connection channel projects from an end portion of the outer tube of the double tube.

16. The liquid ejection device according to claim 5, further comprising a suction device configured to suck the liquid through the suction tube.

17. The liquid ejection device according to claim 1, wherein the pulsed-flow generating section changes a volume of the liquid chamber.

18. The liquid ejection device according to claim 1, wherein the pulsed-flow generating section generates air bubbles in the liquid chamber.

19. A medical apparatus in which the liquid ejection device according to claim 1 is used.
